(19) United States
(12) Patent Application Publication
(10) Pub. No.: US 2018/0001012 A1
(43) Pub. Date: Jan. 4, 2018

(54) Portable Lung Assist Device

(71) Applicants: THE REGENTS OF THE UNIVERSITY OF CALIFORNIA, Oakland, CA (US); THE UNITED STATES GOVERNMENT REPRESENTED BY THE DEPARTMENT OF VETERANS AFFAIRS, Washington, DC (US)

(72) Inventor: Abbas Ardehali, Los Angeles, CA (US)

(21) Appl. No.: 15/536,730

(22) PCT Filed: Dec. 16, 2015
(86) PCT No.: PCT/US15/66092

§ 371 Date: Jun. 16, 2017

Related U.S. Application Data
(60) Provisional application No. 62/092,387, filed on Dec. 16, 2014.

(51) Int. Cl. A61M 1/16; A61M 25/00; A61M 1/26; A61M 1/36

(52) U.S. Cl. CPC A61M 1/1698; A61M 1/3652; A61M 25/0026; A61M 25/005; A61M 1/267; A61M 2205/8206; A61M 2209/088; A61M 2230/20; A61M 2205/3334

(57) ABSTRACT

The present invention relates to a portable lung assist device. In one embodiment, the portable lung assist device comprises an integrated oxygenator, blood pump, and cannula. In one embodiment, the portable lung assist device of the present invention does not require an oxygen tank, but instead can provide oxygen to a subject's blood from ambient air. In one embodiment, at least a portion of the portable lung assist device, for example the cannula can be implantable. In one embodiment, the cannula of the device of the present invention can be inserted in to the subject's pulmonary artery. A method for providing portable lung assistance is also described.

### CROSS-REFERENCE TO RELATED APPLICATIONS

This application claims priority to U.S. provisional application No. 62/092,387 filed on Dec. 16, 2014 incorporated herein by reference in its entirety.

### BACKGROUND OF THE INVENTION

Patients with lung failure are often treated with mechanical ventilation. In some patients, extra-corporeal membrane oxygenation (ECMO) is used to provide oxygen to the patient, or to remove carbon dioxide from the patient, while waiting for the recovery of the patient's own lungs, or as a bridge to lung transplantation. The currently available technology for ECMO requires large beside machines, e.g., a machine that includes a pump, an oxygenator, an air filter, a gas blender, a heat exchanger, and an oxygen tank. The large, heavy nature of these machines can prevent a patient from being able to leave their bed during treatment. Further, most patients on ECMO have to be anesthetized and sedated, leading to deconditioning of these patients.

Some ambulatory ECMO devices for patients with lung failure have been used successfully. However, the ambulatory ECMO devices currently available are still relatively large, heavy, and cumbersome. These devices incorporate the same components from a standard ECMO machine, or marginally smaller versions of these same components, into a system that is placed on a cart or other means for moving the system so that the patient can get out of bed and move around. Therefore, such ambulatory ECMO devices are not truly portable devices that can allow a patient to move around on his or her own, but instead are systems that can allow the patient to change their location, but only with the help of another person that can push the heavy cart that holds the system. Further, these ambulatory ECMO systems require compressed oxygen tanks that must be changed regularly in order to provide a continuous source of oxygen to the system.

In addition, currently available ECMO systems require the presence of trained, licensed perfusion specialists to be physically available around the clock to ensure that these systems are functioning properly. The need for near constant supervision by a specialist greatly reduces the freedom of patients using such systems.

Thus, there is a need in the art for a portable integrated lung assist device that can allow a patient to move around without the help of another person and that does not need require constant supervision by a specialist. Further, there is a need for such a device that does not require an oxygen tank for the oxygen source. The present invention addresses this unmet need in the art.

### SUMMARY OF THE INVENTION

In one embodiment, a portable lung assist device, includes an oxygenator, having a first chamber and a second chamber, the first chamber having an inlet and an outlet, the second chamber having an inlet and an outlet, and a membrane separating the first chamber and the second chamber; a means for supplying a sweep gas to the inlet of the first chamber of the oxygenator; a cannula, having a first lumen and a second lumen; and a pump; where the first lumen is connected to the pump, the pump is connected to the inlet of the second chamber of the oxygenator via a third lumen, and the second lumen is connected to the outlet of the second chamber of the oxygenator; where when the first lumen and second lumen are also connected to a blood vessel of a subject, blood can flow from the subject through the first lumen, through the pump, through the third lumen, through the second chamber of the oxygenator, and through the second lumen back into the subject; and where the subject's blood can be oxygenated via transfer of oxygen across the membrane in the oxygenator. In one embodiment, the sweep gas is air. In one embodiment, at least a portion of the cannula is surgically implantable. In one embodiment, the means for supplying a sweep gas to the oxygenator is a fan. In one embodiment, the pump is a centrifugal pump. In one embodiment, the pump is an axial pump. In one embodiment, an air filter for filtering the sweep gas is included. In one embodiment, a power source is included. In one embodiment, a flow sensor for measuring the rate of blood flow through the device is included. In one embodiment, at least one sensor for measuring the oxygenation level of blood in the device is included. In one embodiment, at least one lumen of the cannula is inserted into the subject's pulmonary artery. In one embodiment, the cannula includes the first lumen in fluid communication with at least one cannula opening and the second lumen in fluid communication with at least one cannula opening, wherein at least one of the openings of the cannula is positioned along the length of the first lumen such that the opening is positioned in the patient's right ventricle when inserted into a patient, and wherein at least one of the openings of the cannula is positioned along the length of the second lumen such that the opening is positioned in the patient's pulmonary artery when inserted into the patient. In one embodiment, at least a portion of the cannula is reinforced with wire.

In one embodiment, a method for providing portable lung assistance includes providing a pump, a power supply electrically coupled to the pump, and an oxygenator for attachment to the body of a patient for portable mobility; positioning a dual lumen cannula tip within a target site in a heart of the patient, wherein the cannula is in fluid communication with the pump and the oxygenator; drawing blood from a patient via the cannula; delivering the blood to a first chamber of the oxygenator so that the blood flows through the first chamber; delivering a sweep gas to a second chamber of the oxygenator, wherein the first and second chambers are separated by a membrane, and wherein carbon dioxide is transferred out of the blood through the membrane and oxygen is transferred into the blood through the membrane as the blood flows through the first chamber; and delivering the oxygenated blood back to the patient via the cannula. In one embodiment, the sweep gas is delivered to the second chamber of the oxygenator from ambient air. In one embodiment, the carbon dioxide is transferred out of the blood is exhausted from the second chamber to ambient air. In one embodiment, pure oxygen is mixed with the sweep gas during delivery. In one embodiment, the cannula further includes the first lumen in fluid communication with at least one cannula opening and the second lumen in fluid communication with at least one cannula opening, wherein at least one of the openings of the cannula is positioned along the length of the first lumen such that the opening is positioned in the patient's right ventricle when inserted into a patient, and wherein at least one of the openings of the cannula is positioned along the length of the second lumen such that the opening is positioned in the patient's pulmonary artery when inserted into the patient. In one embodiment, at least a portion of the cannula is reinforced with wire. In one embodiment, the method includes the step of measuring the rate of blood flow through the first chamber.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is an illustration of the system attached to a human body with the cannula tip advanced to a target site within the heart according to one embodiment.

FIG. 2 is a system diagram of the device according to one embodiment.

FIG. 3 is a diagram of a catheter positioned within a subject's heart according to an exemplary embodiment of the device.

FIG. 4 is a diagram of an oxygenator according to one embodiment.

FIG. 5 is a flow chart of a method for providing portable lung assistance, according to one embodiment.

### DETAILED DESCRIPTION

It is to be understood that the figures and descriptions of the present invention have been simplified to illustrate elements that are relevant for a clear understanding of the present invention, while eliminating, for the purpose of clarity, many other elements found in the field of artificial lungs or lung assist devices. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

The terms “patient,” “subject,” “individual,” and the like are used interchangeably herein, and refer to any animal amenable to the systems, devices, and methods described herein. Preferably, the patient, subject or individual is a mammal, and more preferably, a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The present invention relates to devices and methods for extracorporeal membrane oxygenation (ECMO). The present invention relates to a portable integrated system or device that will support failing human lungs, while allowing the patient to move without the assistance of another person. The present invention addresses the need for a compact system that can be used to oxygenate a patient's blood or to remove carbon dioxide while the patient is waiting for recovery of his or her own lungs, or as a bridge to lung transportation. In one embodiment, the device of the present invention may assist a patient's lungs for 30 days or more. In one embodiment, the present invention relates to veno-venous ECMO. In one embodiment, the present invention relates to arterio-venous ECMO.

In one embodiment, the device of the present invention comprises a pump, an oxygenator, a means for supplying air or oxygen to the oxygenator, and a surgically implantable catheter for the removal and return of blood from the patient. In one embodiment, the device further comprises a heat exchanger. In another embodiment, the device further comprises at least one sensor, for example a flow sensor or an oxygen sensor. In yet another embodiment, the device further comprises an oxygen source, preferably a low-weight, portable oxygen source.

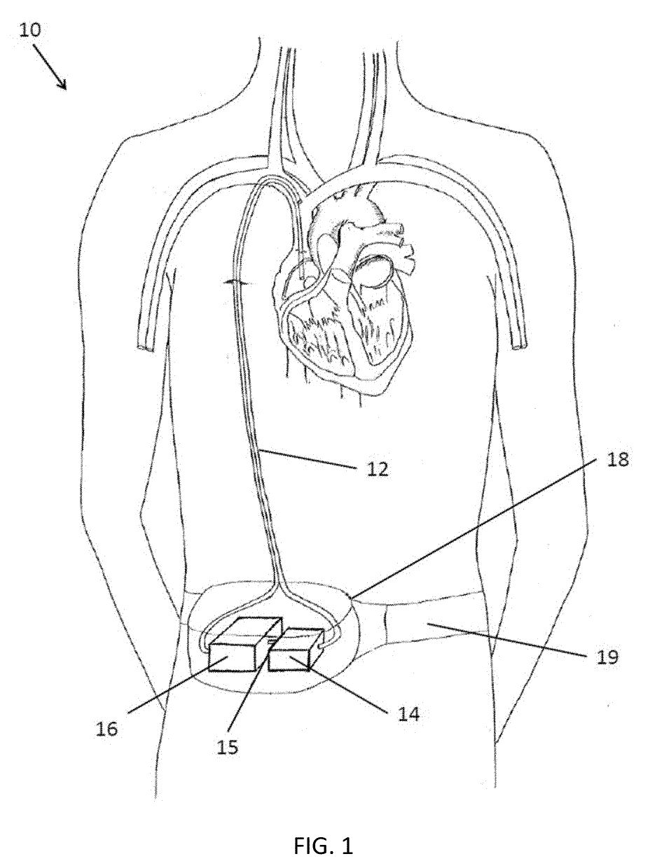

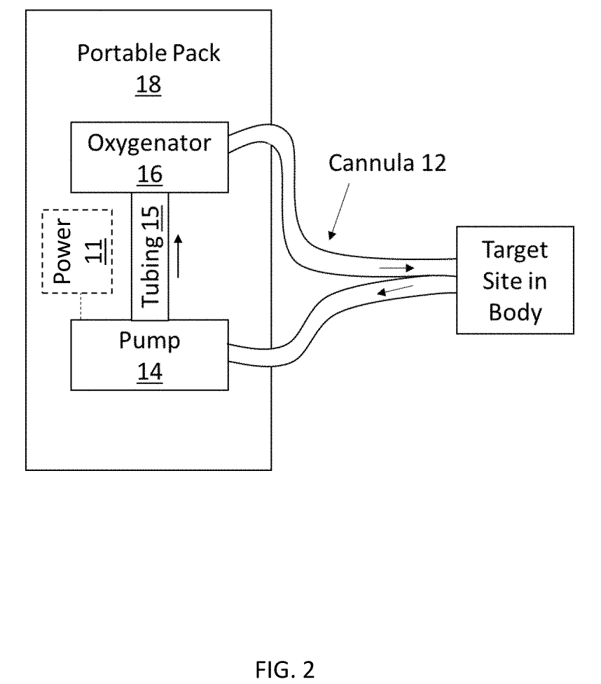

Referring to FIGS. 1 and 2, the lung assist device 10 of the present invention can be used to provide lung support to a subject as follows. Blood is drawn from a subject through a cannula 12, having a first lumen and a second lumen, with the assistance of a pump 14. The pump 14 (and optionally the oxygenator 16) is electrically coupled to and powered by a portable power supply 11, such as rechargeable battery. Pump 14 pushes the blood through another cannula or a piece of tubing 15, into an oxygenator 16, wherein oxygen is transferred into the blood and/or carbon dioxide is removed from the blood. The oxygenated blood is then returned back into the subject via the second lumen in cannula 12. In a preferred embodiment, lung assist device 10 is compact and portable, such that all components of the device other than cannula 12 can be completely contained within a pack 18 that can be attached to a strap or belt 19 for affixing to the user, i.e., the lung assist device is “wearable.” In one embodiment, the lung assist device of the present invention is not wearable. In such an embodiment, all components of lung assist device 10, other than cannula 12, can be sized such that they can be placed inside a portable container, for example a container suitable for carrying by the subject, or a container that can be placed on wheels for easy portability.

In a preferred embodiment, the circuit formed by the cannula, pump, and oxygenator has a relatively small volume, and the blood traveling through the circuit is outside of the subject for only a relatively short amount of time, thereby eliminating the need for a heat exchanger or other component to maintain the temperature of the blood within a range acceptable for medical use. The lack of a need for a heat exchanger enables the device of the present invention to be smaller and, therefore, more portable than ECMO devices currently available. However, in one embodiment, the device of the present invention may include a heat exchanger to ensure that the subject's blood is maintained within an acceptable temperature range.

Further, in a preferred embodiment, the oxygenator of the device of the present invention includes a fan component that pushes air through the oxygenator in order to oxygenate the subject's blood. Accordingly, the system or device of the present invention does not require a separate oxygen source to provide oxygen to the subject's blood, but instead uses ambient air in the environment surrounding the device. The lack of a need for an oxygen source also enables the present invention to be smaller and, therefore, more portable than ECMO devices currently available. However, the device of the present invention may include a separate oxygen source, such as an oxygen tank, to provide increased oxygen concentration to the oxygenator in cases where a higher oxygen concentration than that available in the ambient air is needed.

The device of the present invention includes a means for withdrawing blood from the patient, then returning oxygenated blood to the patient. In various embodiments, the device of the present invention comprises at least one cannula or catheter that is used to remove blood from the patient in order to circulate the blood through an extracorporeal circuit for oxygenation, before returning the blood to the patient. In one embodiment, a portion of the catheter is surgically implanted in the patient. In one embodiment, the catheter is a dual lumen catheter wherein a first lumen is used for removing blood from the subject and a second lumen is used for returning oxygenated blood to the subject.

In one embodiment, the means for withdrawing and returning blood is a single-site venovenous cannulation, wherein a dual-lumen cannula is inserted into the subject. In one embodiment, the dual-lumen cannula is inserted into the jugular vein, extending through the right atrium and into the inferior vena cava. In such an embodiment, venous blood is withdrawn from the vena cava via at least one port in a first lumen of the catheter, while oxygenated blood is returned to the patient's right atrium via at least one port in a second lumen. In another embodiment, a dual-lumen cannula can be inserted into the subject's pulmonary artery. In such an embodiment, oxygenated blood is returned to the subject's pulmonary artery. In various embodiments, the ports in the first lumen and second lumen of the dual-lumen cannula are positioned to reduce circulation of blood directly between the two lumen.

Alternatively, the means for withdrawing and returning blood can be a two-site venovenous cannulation. In one embodiment, a first cannula is inserted in the jugular vein, extending into the right atrium, while a second cannula is inserted into the femoral vein, extending into the inferior vena cava. In such an embodiment, blood is withdrawn via the femoral cannula into the device, then oxygenated blood is reinfused into the patient via the jugular cannula.

In another embodiment, the means for withdrawing and returning blood is a single-site or two-site arterio-venous cannulation. In such an embodiment, the first cannula or lumen, used for returning oxygenated blood to the subject, is inserted into the subject's pulmonary artery. In one embodiment, the second cannula or lumen, used for withdrawing blood from the subject for oxygenation, can be inserted into any blood vessel as would be understood by a person skilled in the art.

An exemplary Cannula known in the art that can be used for the device of the present invention is the Avalon ELITE bi-caval dual lumen catheter. Other exemplary cannulae useful in the device of the present invention are described by Shorey (U.S. patent application Ser. No. 12/145738); Richardson et al. (U.S. Pat. No. 8,118,723); and Reichenbach et al. (U.S. Pat. No. 8,231,519 and U.S. patent application Ser. No. 13/561,197), all of which are incorporated herein by reference in their entirety.

In one embodiment, the lung assist device of the present invention comprises at least one quick-connect mechanism for removably connecting components of the device together. In one embodiment, a surgically implanted cannula of the device may comprise a quick-connect port outside the subject's body for connecting the blood pump or other component of the device to the cannula. In such an embodiment, the surgically implanted cannula can be disconnected from the other components of the device, for example, when one or more components of the device need to be replaced or when oxygenation of the subject's blood is not required. Such a quick-connect mechanism would be suitable for medical applications, and would allow the cannula to be kept in place in the subject for later use, thereby eliminating the need to remove a surgically implanted portion of the device of the present invention. In one embodiment, the quick-connect mechanism further comprises a seal mechanism for isolating the internal lumen of the cannula from the outside environment, thereby eliminating the risk of blood loss and reducing the risk of infection in the subject. An example of a quick-connect mechanism suitable for use in the present invention is described by Dormanen et al. (PCT/US2013/025703)

The device of the present invention includes a pump that is used to maintain the desired flow rate of blood through the device. In various embodiments, the pump of the device of the present invention can supply enough head pressure to overcome the resistance of an oxygenator and any tubing or cannula used to direct the flow of blood in the device. In various embodiments, the pump is any type of pump suitable for use with human blood, as understood by a person skilled in the art. In one embodiment, the pump is a centrifugal pump. In another embodiment, the pump is a pneumatic pump. In yet another embodiment, the pump is an axial or impeller pump.

The pump component can be used to provide a flow rate of blood through the device of the present invention that is typically in the range of 1 to 5 liters per minute (L/min). In a preferred embodiment, the maximum flow capacity of the pump is about 2.5 L/min. In various embodiments, the pump of the present invention generates enough pressure to circulate blood through the device without causing significant hemolysis. In one embodiment, the pressure change (AP) across the pump is at least 40 mm Hg in order to achieve the desired flow rate of blood through the device of the present invention. In a preferred embodiment, the AP is about 50 mm Hg. However, the pump used for the device of the present invention is not limited to the values for flow rate and/or AP described herein, and can be any value as would be understood by a person skilled in the art.

Pumps that can be used in the lung assist device of the present invention are known in the art. Such pumps can be sold separately commercially, or can be incorporated into a device having other components, such as a Left Ventricular Assist Device (LVAD). Exemplary pumps that can be used in the present invention can be found in the Thoratec HEARTMATE II LVAD, Thoratec Paracorporeal Ventricular Assist Device (PVAD), or Thoratec Implantable Ventricular Assist Device (IVAD). Other exemplary blood pumps are described in McBride et al. (U.S. Pat. No. 7,841,976); Tansley et al. (U.S. Pat. No. 8,366,599); and Campbell et al. (U.S. Pat. No. 8,535,211), all of which are incorporated herein by reference in their entirety.

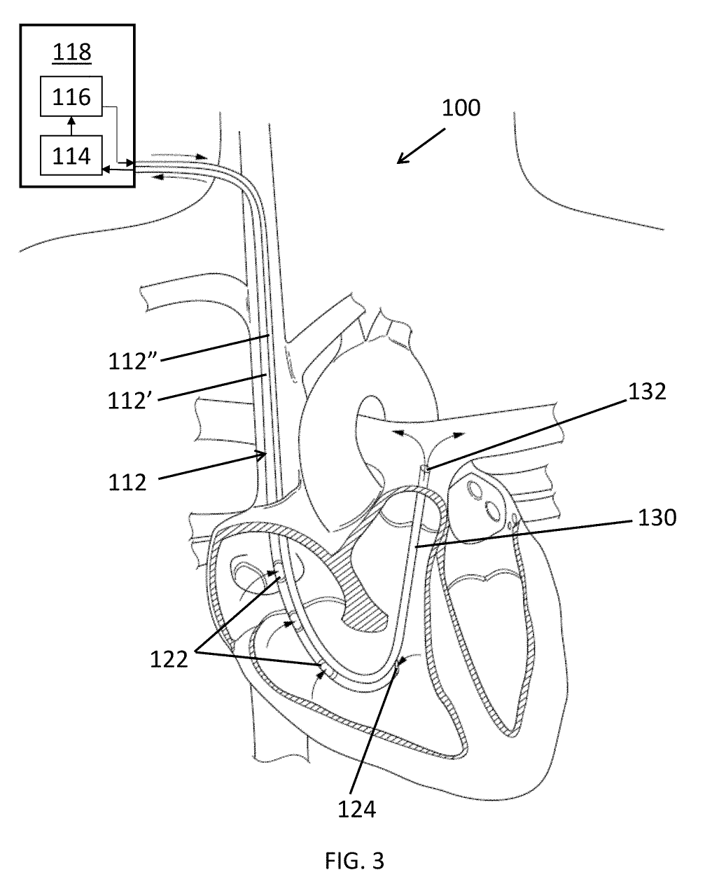

In one embodiment, the pump is used with a specialized catheter, such as that described in International Application No. PCT/US2015/027334, filed on Apr. 23, 2015 and incorporated herein by reference in its entirety. With reference now to FIG. 3, the lung assist device 100 draws blood from a subject through a cannula 12, having a first lumen and a second lumen, with the assistance of a pump 114. The pump 114 pushes the blood through another cannula or a piece of tubing, into an oxygenator 116, wherein oxygen is transferred into the blood and/or carbon dioxide is removed from the blood. The oxygenated blood is then returned back into the subject via the second lumen in cannula 112. The lung assist device 100 is compact and portable, such that all components of the device other than cannula 12 can be completely contained within a pack 118 for portable mobility. In certain embodiments, some or all of the units can be positioned on a portable stand or card, instead of within pack 118. In certain embodiments, some or all of the units are positioned on devices known in the art that allow the units to be portable and afford the patient portable mobility. Cannula 112 includes a first tube 112, having a lumen 112′ for directing the inflow of blood from the patient to the pack 118, and a second tube 112″, having a lumen for directing the outflow of blood from pack 118 back into the patient. The first tube 112′ can be positioned so that it extends through the tricuspid valve and into the right ventricle. First tube 122′ has one or more openings 122 in the wall of the tube for draining blood from the superior vena cava, right atrium, and/or right ventricle into the lumen of first tube 112′. First tube 112′ also has an opening 124 at or near its distal tip, which can be used to drain blood specifically from the right ventricle. Second tube 112″ of cannula 112 is positioned so that it extends through the tricuspid valve and right ventricle, and through the pulmonary valve into the pulmonary artery, so that an opening 132 at or near the tip of second tube 112″ can be used to send oxygenated blood directly into the pulmonary artery. In one embodiment, second tube 130 can also include one or more openings in the wall of the tube, particularly near the tip of the tube, instead of or in addition to opening 132. In one embodiment, at least a portion of one or both tubes of the cannula 112 are reinforced with wire. The wire reinforcement can be designed accordingly so that the cannula can be suitably advanced into position within the patient, and so that the catheter is stabilized in the optimal location, once positioned. Accordingly, the catheter of the present embodiment may include portions or regions having the desired stiffness, rigidity or flexibility necessary for proper insertion into the subject and subsequent functionality. Advantageously, when used with a cannula as described above, recirculation of oxygenated blood is minimized, and pump efficiency is increased. Further, the position of the cannula is more stable, which is of high importance in a system that encourages mobility of the patient. Normally, the more the patient moves their upper body, specifically the head and neck region, the higher the chance that the cannula can shift, migrate or dislodge from the desired position in eh heart. Movement of the catheter against a vessel wall can also cause catheter openings under negative pressure (e.g. lumen 112′ and openings 122) to suck up against an interior vessel wall, blocking inflow of blood. According to embodiments that use the cannula described above, movement of the catheter is minimized. Further, bending and kinking of the cannula is minimized, and the cannula therefore maintains a larger cross-sectional area. Thus, maximized flow rates through the cannula are maintained, while preventing high pressures that can lead to red blood cell shearing and hemolysis. Since the cannula is capable of effectively and efficiently draining blood from both the right atrium and right ventricle, a system according to embodiments described herein can have a smaller and lighter pump that is capable of being powered from a portable power source without compromising the supply of requisite flow rates, enabling the system to be entirely portable.

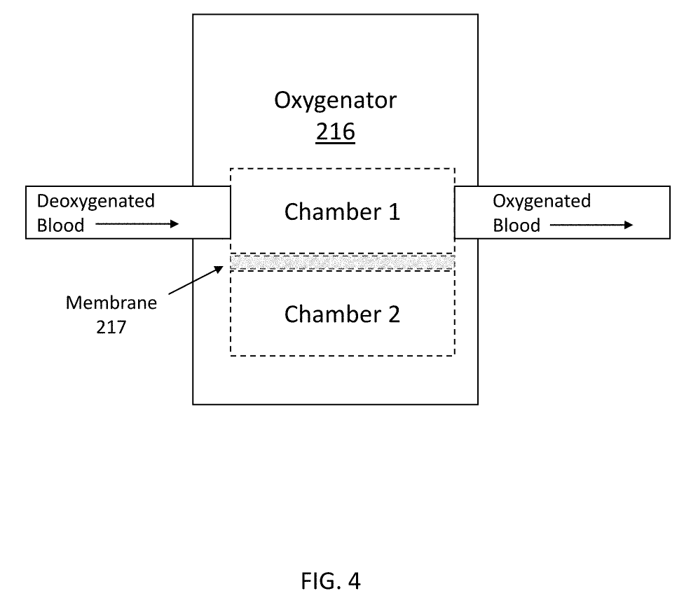

The device of the present invention includes an oxygenator for transferring oxygen from an oxygen source to the subject's blood. In one embodiment, as shown in FIG. 4, the oxygenator 216 comprises a membrane 217 that allows oxygen to diffuse into the blood while also allowing carbon dioxide to diffuse out of the blood. In such an embodiment, the oxygenator comprises two chambers separated by a semipermeable membrane. A pump delivers venous blood, or blood in need of oxygenation or carbon dioxide removal from another location in the subject, from the subject to the oxygenator, wherein the venous blood flows through the first chamber of the oxygenator. A sweep gas is simultaneously delivered to the second chamber of the oxygenator. As the blood flows through the first chamber, gas exchange occurs across the membrane separating the first and second chambers. In such a gas exchange, oxygen is transferred from the sweep gas in the second chamber into the blood in the first chamber. Further, carbon dioxide present in the blood will be transferred out of the blood into the sweep gas in the second chamber. The blood that exits the first chamber is returned to the patient, while the gas that exits the second chamber can be sent to the ambient environment, or to an exhaust vent in the room. In one embodiment, the oxygenator has a minimum surface area that corresponds to the delivery of oxygen to the patient's blood at a rate of about 180 cc/min.

In one embodiment, the sweep gas comprises fresh air that is delivered to the second chamber via a fan. In another embodiment, the sweep gas comprises a mixture of air and oxygen that is blended prior to being delivered to the second chamber. In such an embodiment, a feed gas, such as pure oxygen, can be combined with ambient air to form the sweep gas. Further, in such an embodiment, the device of the present invention may comprise additional components, such as an air blender or mixer, and a sweep gas pump to pump the mixed sweep gas to and through the second chamber of the oxygenator.

The concentration of carbon dioxide and oxygen in the blood exiting the oxygenator, i.e., the blood that will be returned to the patient, is primarily determined by the partial pressures of the respective gases in the blood and the sweep gas, and the characteristics of both the membrane and the first and second chambers. For example, if the surface area of the membrane is relatively large compared to the volume of the first chamber, a relatively high rate of gas diffusion across the membrane can occur. Additionally, if the difference in partial pressure of a gas species across the membrane, i.e., the difference in partial pressure between the first and second chambers, is significantly high, then a relatively high rate of gas diffusion can occur. Other variables that can affect the oxygen uptake and carbon dioxide elimination in the blood in the second chamber are the flow rate of sweep gas through the first chamber, the flow rate of blood through the second chamber, and the absolute pressure inside the first chamber. In one embodiment, the sweep gas can flow in a direction countercurrent to the flow of blood. In another embodiment, the sweep gas can flow concurrently to the flow of blood.

In various embodiments, the oxygenator of the device of the present invention can be an exemplary oxygenator known in the art. Exemplary oxygenators include the Medtronic AFFINITY FUSION oxygenation system, Medtronic AFFINITY NT oxygenation system, Medtronic AFFINITY PIXIE oxygenation system, Medtronic MINIMAX PLUS oxygenation system, Maquet QUADROX oxygenation system, Sorin KIDS oxygenator, Sorin APEX oxygenator, Sorin BMR oxygenator, Sorin PRIMO2X oxygenator, Sorin SYNERGY oxygenator, or Sorin SYNTHESIS oxygenator. In one embodiment, the oxygenator can be a membrane ventilator. Exemplary membrane ventilators include the Novalung MINILUNG membrane ventilator, the Novalung iLA membrane ventilator, and the Novalung XLUNG membrane ventilator. In one embodiment, the pump and oxygenator of the device of the present invention can be an integrated unit, such as the blood-pump oxygenator described by Gellman et al. (U.S. Pat. No. 8,496,874).

In various embodiments, operating parameters in the lung assist device of the present invention, such as the sweep gas flow rate, blood flow rate, and gas pressure in the oxygenator, can be optimized to achieve the desired performance. In one embodiment, the sweep gas flow rate can be up to about 15 L/min. In one embodiment, the gas pressure can be up to about 30 mm Hg. In one embodiment, the blood flow rate can be up to about 5 L/min. However, the operating parameters of the device of the present invention are not limited to the values listed herein. Generally, a relatively low blood flow rate through the oxygenator requires a correspondingly high gas flow rate and/or gas pressure to achieve sufficient blood oxygenation. Conversely, a relatively high blood flow rate can reduce the gas flow rate and/or gas pressure values required for sufficient blood oxygenation.

Additionally, the operating parameters of the lung assist device of the present invention can be adjusted depending on whether the device is primarily being used to remove carbon dioxide from the blood instead of oxygenation. For example, a relatively low flow rate of blood through the device is needed when carbon dioxide removal, rather than oxygen delivery, is the primary focus of use of the lung assist device.

In one embodiment of the present invention, ambient air, i.e., air in the environment immediately surrounding the device, is used as the sweep gas feed to the oxygenator. Ambient air typically comprises 20.95% oxygen and less than 0.04% carbon dioxide by volume. By using ambient air as the gas feed for the oxygenator, the device of the present invention eliminates the need for an oxygen tank or other source of oxygen, thereby increasing the portability of the device. Alternatively, in another embodiment, ambient air can be mixed with oxygen from an oxygen source prior to be supplied to the oxygenator in order to increase the concentration of oxygen in the gas feed, thereby increasing the rate of oxygen transfer to the blood.

Accordingly, the device of the present invention may include a concentrated oxygen source to supply oxygen to the subject's blood via the oxygenator. In one embodiment, the concentrated oxygen source is an oxygen canister tank, whereby compressed oxygen in the form of a liquid or gas is stored and supplied to the system as needed through a valve. In such embodiment, oxygen from the concentrated oxygen source may be added to ambient air in order to increase the concentration of oxygen in the sweep gas being supplied to the oxygenator.

The device of the present invention may further comprise a power source, or a means for supplying power to the device. In one embodiment, the power source can be a battery, preferably a compact battery pack that is suitable for a portable device. In one embodiment, the battery comprises a lithium battery. In another embodiment, the power source can be provided via a power cord suitable for connecting the device to an electrical outlet, in cases where the patient is waiting for the battery to be recharged, or when the patient desires to remain in a location that is suitably close to an electrical outlet.

In various embodiments, the device of the present invention may comprise additional components that will improve the performance of blood oxygenation and lung assistance. Such components include, but are not limited to: a heat exchanger, at least one sensor, an air filter, and a control panel or other means for controlling the device.

In one embodiment, the device of the present invention may comprise a heat exchanger. In such an embodiment, the heat exchanger is used to maintain the temperature of the subject's blood at or close to the subject's natural body temperature in order to prevent or reduce the potential for causing adverse health effects associated with a decrease in temperature of the blood while the blood is outside the subject's body. In one embodiment, the heat exchanger is small and compact in size in order to maintain portability of the device while serving to minimize the effects of the patient's blood being exposed to ambient, i.e., room temperature. In one embodiment, the heat exchanger is a shell and tube heat exchanger.

In various embodiments, the device of the present invention comprises at least one sensor for measuring variables related to the operation of the device. In one embodiment, the device comprises an oxygen sensor for determining the level of oxygen in the subject's blood. In another embodiment, the device comprises a carbon dioxide sensor for determining the level of carbon dioxide in the subject's blood. In one embodiment, the oxygen and/or carbon dioxide sensors can be used for measuring the concentration of a gas in the blood entering the device, i.e., pre-oxygenation. In another embodiment, the oxygen and/or carbon dioxide sensors can be used for measuring the concentration of a gas in the blood returning to the patient, i.e., post-oxygenation. In one embodiment, the device comprises at least one sensor for determining the composition of oxygen and/or carbon dioxide in the sweep gas. In one embodiment, the device comprises at least one flow sensor for measuring the flow rate of blood at a desired location in the system. In one embodiment, the device comprises a flow sensor for measuring the flow rate of sweep gas in the oxygenator. In one embodiment, the device comprises temperature sensors for determining the temperature of the blood at a desired location in the device, for example, the temperature of blood in the second lumen as it is being returned to the patient.

In one embodiment, the device comprises an air filter for filtering particulates or other impurities from the gas being supplied to the oxygenator. In one embodiment, the filter is capable of filtering about 95% of particles that are 0.3 microns or larger.

In various embodiments, the device of the present invention may comprise a means for controlling the device, for example, to control variables such as, but not limited to, the flow rate of blood through the device, the flow rate of air through the device, the composition of sweep gas, and the temperature of blood flowing through the device. In one embodiment, the control means is a compact controller integrated with the device, comprising a touch screen or other means for entering and/or displaying data. In another embodiment, the control means may comprise a computer processor integrated with the device that can be controlled via a wireless connection to a computer that is not physically connected to the device.

In one embodiment, integrated software may be used to automatically adjust the flow, pressure, the sweep gas flow, and/or other parameters to optimize and/or meet the patient's physiologic needs. In one embodiment, a wireless remote monitoring system can be included in device of the present invention to allow the subject or a caretaker to monitor the subject and/or the ECMO circuit performance.

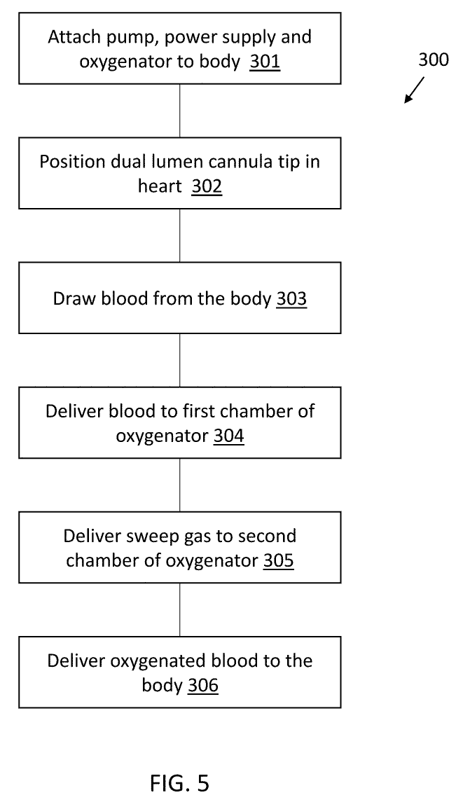

A method for providing portable lung assistance is also provided, with reference now to FIG. 5. In one embodiment, the method includes providing a pump, a power supply electrically coupled to the pump, and an oxygenator to the body of a patient for portable mobility 301. A dual lumen cannula tip is positioned within a target site in a heart of the patient 302, and the cannula is in fluid communication with the pump and the oxygenator. Blood is drawn blood from the patient via the cannula 303, and the blood is delivered to a first chamber of the oxygenator 304 so that the blood flows through the first chamber. A sweep gas is delivered to a second chamber of the oxygenator 305. The first and second chambers are separated by a membrane, carbon dioxide is transferred out of the blood through the membrane and oxygen is transferred into the blood through the membrane as the blood flows through the first chamber. The oxygenated blood is delivered back to the patient via the cannula 306. In one embodiment, the sweep gas is delivered to the second chamber of the oxygenator from ambient air. In one embodiment, the carbon dioxide is transferred out of the blood is exhausted from the second chamber to ambient air. In one embodiment, the pure oxygen is mixed with the sweep gas during delivery. In one embodiment, the cannula further includes the first lumen in fluid communication with at least one cannula opening and the second lumen in fluid communication with at least one cannula opening, wherein at least one of the openings of the cannula is positioned along the length of the first lumen such that the opening is positioned in the patient's right ventricle when inserted into a patient, and wherein at least one of the openings of the cannula is positioned along the length of the second lumen such that the opening is positioned in the patient's pulmonary artery when inserted into the patient. In one embodiment, at least a portion of the cannula is reinforced with wire.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A portable lung assist device, comprising:
an oxygenator, having a first chamber and a second chamber, the first chamber having an inlet and an outlet, the second chamber having an inlet and an outlet, and a membrane separating the first chamber and the second chamber;
a means for supplying a sweep gas to the inlet of the first chamber of the oxygenator;
a cannula, having a first lumen and a second lumen; and
a pump;
wherein the first lumen is connected to the pump, the pump is connected to the inlet of the second chamber of the oxygenator via a third lumen, and the second lumen is connected to the outlet of the second chamber of the oxygenator;
wherein when the first lumen and second lumen are also connected to a blood vessel of a subject, blood can flow from the subject through the first lumen, through the pump, through the third lumen, through the second chamber of the oxygenator, and through the second lumen back into the subject; and
wherein the subject's blood can be oxygenated via transfer of oxygen across the membrane in the oxygenator.

2. The device of claim 1, wherein the sweep gas is air.

3. The device of claim 1, wherein at least a portion of the cannula is surgically implantable.

4. The device of claim 1, wherein the means for supplying a sweep gas to the oxygenator is a fan.

5. The device of claim 1, wherein the pump is a centrifugal pump.

6. The device of claim, wherein the pump is an axial pump.

7. The device of claim 1, further comprising an air filter for filtering the sweep gas.

8. The device of claim 1, further comprising a power source.

9. The device of claim 1, further comprising a flow sensor for measuring the rate of blood flow through the device.

10. The device of claim 1, further comprising at least one sensor for measuring the oxygenation level of blood in the device.

11. The device of claim 1, wherein at least one lumen of the cannula is inserted into the subject's pulmonary artery.

12. The device of claim 1, wherein the cannula further comprises:
the first lumen in fluid communication with at least one cannula opening and the second lumen in fluid communication with at least one cannula opening, wherein at least one of the openings of the cannula is positioned along the length of the first lumen such that the opening is positioned in the patient's right ventricle when inserted into a patient, and wherein at least one of the openings of the cannula is positioned along the length of the second lumen such that the opening is positioned in the patient's pulmonary artery when inserted into the patient.

13. The device of claim 12, wherein at least a portion of the cannula is reinforced with wire.

14. A method for providing portable lung assistance comprising:
providing a pump, a power supply electrically coupled to the pump, and an oxygenator for attachment to the body of a patient for portable mobility;
positioning a dual lumen cannula tip within a target site in a heart of the patient, wherein the cannula is in fluid communication with the pump and the oxygenator;
drawing blood from a patient via the cannula;
delivering the blood to a first chamber of the oxygenator so that the blood flows through the first chamber;
delivering a sweep gas to a second chamber of the oxygenator, wherein the first and second chambers are separated by a membrane, and wherein carbon dioxide is transferred out of the blood through the membrane and oxygen is transferred into the blood through the membrane as the blood flows through the first chamber; and
delivering the oxygenated blood back to the patient via the cannula.

15. The method of claim 14, wherein the sweep gas is delivered to the second chamber of the oxygenator from ambient air.

16. The method of claim 14, wherein the carbon dioxide is transferred out of the blood is exhausted from the second chamber to ambient air.

17. The method of claim 14, wherein pure oxygen is mixed with the sweep gas during delivery.

18. The method of claim 14, wherein the cannula further comprises:
the first lumen in fluid communication with at least one cannula opening and the second lumen in fluid communication with at least one cannula opening, wherein at least one of the openings of the cannula is positioned along the length of the first lumen such that the opening is positioned in the patient's right ventricle when inserted into a patient, and wherein at least one of the openings of the cannula is positioned along the length of the second lumen such that the opening is positioned in the patient's pulmonary artery when inserted into the patient.

19. The method of claim 18, wherein at least a portion of the cannula is reinforced with wire.

20. The method of claim 14 further comprising:
measuring the rate of blood flow through the first chamber.
